(19) United States
(12) Patent Application Publication
(10) Pub. No.: US 2026/0248659 A1
(43) Pub. Date: Aug. 27, 2026

(54) Menstrual insert

(71) Applicant: Veronica Belletto, New Orleans, LA (US)

(72) Inventor: Veronica Belletto, New Orleans, LA (US)

(21) Appl. No.: 18/831,486

(22) Filed: Feb. 24, 2025

(51) Int. Cl. A61F 13/20; A61F 13/26; A61F 13/34

(52) U.S. Cl. CPC A61F 13/2022; A61F 13/2074; A61F 13/266; A61F 13/34

(57) ABSTRACT

A menstrual insert includes a flexible, ultra-absorbent pad that conforms to the vaginal anatomy, including the cervix. The insert is designed to be leak-resistant, body-adaptive, and undetectable during wear. A retrieval loop or string allows for safe removal. The insert may be infused with medicinal agents for pH balance or anti-inflammatory relief. It can be inserted manually or using an applicator with a plunger mechanism. The applicator facilitates precise placement and easy removal, ensuring maximum comfort and effectiveness.

### BACKGROUND OF THE INVENTION

Menstrual care products have evolved significantly over the years, offering a variety of options for individuals seeking comfort, convenience, and reliability. Traditional products such as pads and tampons have limitations in leakage control, comfort, and sustainability. The present invention relates to an improved menstrual insert designed to address these concerns by providing enhanced absorption, fit, and user comfort.

Conventional menstrual products may allow fluid bypass under certain conditions, which can cause inconvenience. Additionally, existing products may shift during movement, leading to discomfort or leakage. Some users attempt to retain a menstrual device during activities such as sexual intercourse, but conventional options are not designed for this purpose, leading to potential risks of displacement or ineffective absorption.

Accordingly, there is a need for a menstrual insert that provides secure placement, improved absorption, and enhanced comfort. The present invention introduces a disk-shaped, ultra-absorbent insert that conforms to the cervix while remaining undetectable during wear.

### SUMMARY OF THE INVENTION

The present invention provides a menstrual insert designed to conform to the vaginal anatomy, including the base of the cervix, ensuring optimal absorption and comfort. The insert is composed of a medical-grade, body-safe material that mimics the texture and elasticity of vaginal tissue.

One objective of the invention is to provide a leak-resistant design that adapts to body movement and temperature, offering increased security. Another objective is to incorporate antimicrobial properties to help prevent infections while maintaining a healthy pH balance.

The menstrual insert may be used with or without an applicator. In the preferred embodiment, the insert includes a string or loop mechanism for easy removal. The insert can also be infused with medicinal or pH-balancing agents to enhance comfort and hygiene.

### BRIEF DESCRIPTION OF THE DRAWINGS

FIG. 1 is a perspective view of the absorbent menstrual insert.

FIG. 2 is a bottom view of the insert.

FIG. 3 is a front, plan view illustrating the curvature of the insert.

FIG. 4 is an isolated view of the applicator.

### DETAILED DESCRIPTION OF THE INVENTION

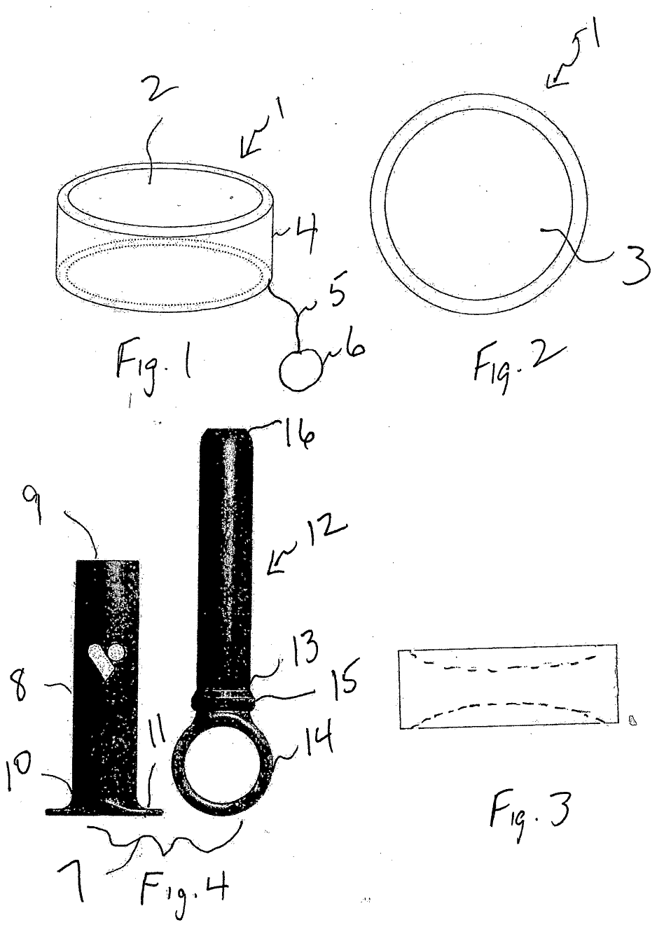

Referring to FIG. 1, the menstrual insert (10) comprises a flexible, ultra-absorbent core (12) that expands upon insertion, ensuring a secure fit. The outer layer (14) is coated with a biocompatible, moisture-wicking material that enhances absorption while minimizing bacterial growth.

In one embodiment, the menstrual insert is infused with natural anti-inflammatory agents to reduce discomfort and irritation. In another embodiment, the insert is designed with a retrieval loop (16) to facilitate easy removal without a traditional string.

The insert may be inserted using a user's finger or an accompanying applicator (FIG. 4). The applicator (20) includes a hollow barrel (22) with an open upper end (24), an open lower end (26), and a hollow interior (28). The upper end is slightly tapered to facilitate insertion into and removal from the vaginal canal.

A plunger (30) is telescopically received within the lower end of the barrel. The plunger (30) comprises an elongated tube (32) with two opposing closed ends. A finger ring (34) at the first end allows for easy operation, while an annular stop member (36) prevents excessive penetration of the plunger into the barrel.

To apply the absorbent pad, the user compresses the insert into the barrel interior, inserts the applicator into the vaginal canal, and depresses the plunger to deliver the pad to the cervix or fornix. If engaging in routine activity, the wearer may position the pad a few inches within the vaginal cervix. If planning to engage in sexual activity, the user positions the pad at the fornix.

The above-described device is not limited to the exact details of construction and enumeration of parts provided herein. Furthermore, the size, shape, and materials of construction of the various components can be varied.

1. A menstrual insert comprising a flexible, ultra-absorbent pad designed to conform to the vaginal anatomy, including the base of the cervix, and constructed of a material that mimics vaginal tissue.

2. The menstrual insert of claim 1, further comprising a retrieval mechanism including a string or loop to facilitate removal.

3. The menstrual insert of claim 1, wherein the pad is infused with a medicinal agent for pH balance or anti-inflammatory benefits.

4. The menstrual insert of claim 1, wherein an upper surface is concave to conform to the base of the cervix.

5. The menstrual insert of claim 1, further comprising an applicator for insertion, said applicator including: a hollow barrel having an open upper end, an open lower end, and a hollow interior; and a plunger telescopically received within the barrel.

6. The menstrual insert of claim 5, wherein the upper end of the barrel is tapered for improved insertion and removal.

7. The menstrual insert of claim 6, wherein the lower end of the barrel includes a flange that is grasped by the user.

8. The menstrual insert of claim 5, wherein the plunger includes a finger ring for user control.

9. The menstrual insert of claim 5, further comprising a stop member adjacent to the first closed end of the plunger to limit insertion depth.

10. The menstrual insert of claim 8, wherein the opposing closed end of the plunger is tapered for seamless insertion into the barrel.

11. The menstrual insert of claim 1, wherein the pad is constructed with medical-grade foam.
